(19) United States
(12) Patent Application Publication
(10) Pub. No.: US 2003/0176806 A1
(43) Pub. Date: Sep. 18, 2003

(54) Method and system for an intelligent supervisory control system

(76) Inventors: Jaime A. Pineda, San Diego, CA (US); Brendan Z. Allison, La Jolla, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 10/376,676

(22) Filed: Feb. 26, 2003

Related U.S. Application Data
(60) Provisional application No. 60/360,153, filed on Feb. 26, 2002.

(51) Int. Cl. A61B 5/04

(52) U.S. Cl. 600/544

(57) ABSTRACT

An intelligent supervisory control system or neuro-user interface (NUI), and method that utilize bioelectric state of mind or cognitive profile to control electronic and mechanical resources in an environment, such as in a 3-D PC or console game, a simulation or virtual environment, a cockpit, automobile, home, or surgical theatre. The interface comprises means for acquiring the brain signals of a user or subject, which are converted into a digital stream and mathematically processed to define an electrical state of the mind or cognitive profile of the user. Incorporating microprocessor-based software and storage facilities, the interface dynamically maps the cognitive profile onto multiple functions, which are adaptable for actuating microprocessor commands. In conjunction with other standard input devices such as mouse, keyboard, or joystick, the intelligent supervisory control interface of the present invention thus provides a user with the maximal degrees of freedom for the control of the resources (or electrical and mechanical devices) in the environment.

### CROSS REFERENCE TO RELATED APPLICATION

This application claims priority to provisional patent application serial No. 60/360,153 filed on Feb. 26, 2002.

### FIELD OF THE INVENTION

The present invention relates to intelligent supervisory control systems. In particular, this invention relates to a system and the use of motor and non-motor bioelectrical signals for the control of electronic and mechanical devices.

### BACKGROUND OF THE INVENTION

It has been recognized that the use of real-time graphics, multimedia, and ubiquitous computing, combined with future developments in 3-D representation, is creating complex microprocessor environments in which visual information overload is common and in which the usual modes of communicating with a device such as a computer using a keyboard or mouse are slow and inefficient.

In certain situations, the use of brain signals can be applied to facilitate human-device interactions for actuation of electronic and/or mechanical devices. These types of interactions provide for a rapid control system, which not only increases the degrees of freedom that a user has over an environment, but also makes individual interactions with devices a more seamless, enjoyable, and productive experience.

There is a need to develop more natural and intuitive interfaces that can recognize innate human skills, such as handwriting, gesture, and speech. Despite recent improvements in biological sensor technology, signal processing methodology, pattern recognition techniques, and high-speed computational algorithms, the development and use of a rapid neural user interface to increase degrees of freedom in controlling an environment is non-existent or quite limited. Though there is existing use of neural user interface technology for medical applications such as communication for disabled individuals and control of prosthetic devices, and for passive (i.e., one-way) monitoring of vigilant and attentional states, prior art lacks rapid bi-directional control over an environment.

Furthermore, while existing prior art may use either the spontaneous EEG or evoked potential, it focuses on a single outcome function, which relates the brain signal to a single outcome event. The inference of such an outcome from a single brain signal fails to take into account the dynamics of the different brain signals (neurodynamics), singularly, or in combination, as a means to control multiple functions associated with different resources in such an environment. Thus, though such use of the brain signal can effect as a substitute for the physiological human interaction with the environment, for example, using the brain signal to operate a computer keyboard, one's control over the resources of the environment is quite limited. It is also devoid of the dynamics of the state of the mind of the user while interacting with the environment. Such state of the mind dynamics, or neurodynamics, are considered to result from the non-linear combination of different brain signals, which are key attributes of a user's overall immersed experience associated with the rapid transfer of brain signals over the controls of the environment.

Accordingly, it would be desirable to have a system that incorporates non-linear neurodynamics as part of a neural user interface (NUI™) control to enable dynamic mapping between the neural signals and outcome events. Furthermore, it would be desirable for the NUI to recognize the functional significance of the various relevant components of the brain signals measured in the form of bioelectrical signals. The present invention addresses these fundamental attributes. In particular, the present invention incorporates a state of the mind, as characterized by the non-linear combination of bioelectric signals, as a means to control the resources of the environment, adding improved efficiency and immersion in the overall experience of the user.

### SUMMARY OF THE INVENTION

The present invention provides an intelligent supervisory control system, or neuro-user interface, and method that utilize bioelectric state of the mind or cognitive profile to control electronic and mechanical resources in an environment, such as in a 3-D PC or console game, a simulation or virtual environment, a cockpit, automobile, home, or surgical theatre. The neuro-user interface comprises means for acquiring the brain signals of a user or subject, which are converted into a digital stream and mathematically processed to define an electrical state of the mind or cognitive profile of the user. Incorporating microprocessor-based software and storage facilities, the interface dynamically maps the cognitive profile onto multiple functions, which are adaptable for actuating microprocessor commands. In conjunction with other standard input devices such as mouse, keyboard, or joystick, the intelligent supervisory control system of the present invention provides a user with the maximal degrees of freedom for the control of the resources (or electrical and mechanical devices) in the environment.

In addition to providing maximal freedom of control, the interface is adaptable to process the brain signals into microprocessor commands in real time and to allow closed-loop feedback that does not require constant monitoring of the input. The invention incorporates adaptive pattern recognition algorithms that allow for automated learning, whereby the system learns to recognize a user's specific brain signals over time. These brain signals include spontaneous electroencephalogram (EEG) rhythms in combination with event-related potentials and steady state responses related to motor and non-motor thought patterns that are relevant to the user. For mapping, inputs representing values from several types of brain signals can be recorded (EEG rhythms, ERPs, SSVERs).

The present invention incorporates spontaneous EEG rhythms, for example, those measured over the sensorimotor cortex, time-locked responses to external events, and event-related potentials (ERPs), as well as steady state visual evoked responses, in a rapid bi-directional neural user interface. The invention enables the user to learn to control the magnitude and effect of these categories of brain signals within a short period of time. By controlling these signals, such as by producing or blocking them individually or in combination, the present invention dynamically maps the levels of brain activities to microprocessor commands. The mapping function comprises of inputs and values from one or more several types of brain signals, a combinatorial algorithm, and an association of the specific combination with a particular microprocessor command.

The brain signals obtained from the user are analyzed and adapted for control in a variety of ways. The different dimensions of the signals are decomposed, analyzed, and used. Taking into consideration of the individual differences which occur in baseline measurements of the bioelectric signals and in the degree of control that users learn to exert over their EEG, the learning algorithms ensures that the adaptive nature of the present invention be maximized and that its control be refined over time.

Thus, the invention represents a unique, novel, more natural, intuitive, and hands-free means of communicating with and controlling devices or resources in an environment, which takes into account the non-linear details of neurodynamics and reflects the users' mood and state of the mind.

### BRIEF DESCRIPTION OF THE DRAWINGS

FIG. 1 is a block diagram representing an intelligent supervisory control system, or neural user interface, of the present invention.

FIG. 2 is a flow diagram of a method for brain signal acquisition in accordance with the methods and systems of the present invention.

FIG. 3 depicts a variety of sensor-to-microprocessor links in accordance with the method of this invention.

FIG. 4 lists the variety of brain signals that are captured and used in the present invention to control resources in an environment.

FIG. 5 depicts a diagrammatic view of the decomposition and analysis of brain signals in accordance with the method of this invention.

FIG. 6a depicts a 5s epoch of EEG, the embedded signal (10 Hz, 20 mV; 10 Hz, 10 mV and 20 Hz, 10 mV), and the output of the VEFD.

FIG. 6b depicts a simulated “rhythmic” EEG and the output of the power spectrum and VEFD.

FIG. 7a depicts the raw P300 data recorded at 500 ms presentation speed before ICA reconstitution or “cleanup.”

FIG. 7b depicts the raw P300 data of FIG. 7a post cleanup.

FIG. 8 depicts a flowchart of the learning and pattern recognition analysis of brain signals in accordance with the systems and methods of this invention.

FIG. 9 depicts a flowchart of the computer interface and closed-loop feedback analysis of brain signals in accordance with the systems and methods of this invention.

FIG. 10 depicts an exemplary neural network that can be used by the present invention.

FIG. 11 depicts an exemplary computational algorithm for manipulating input signals according to an embodiment of the present invention.

### DETAILED DESCRIPTION

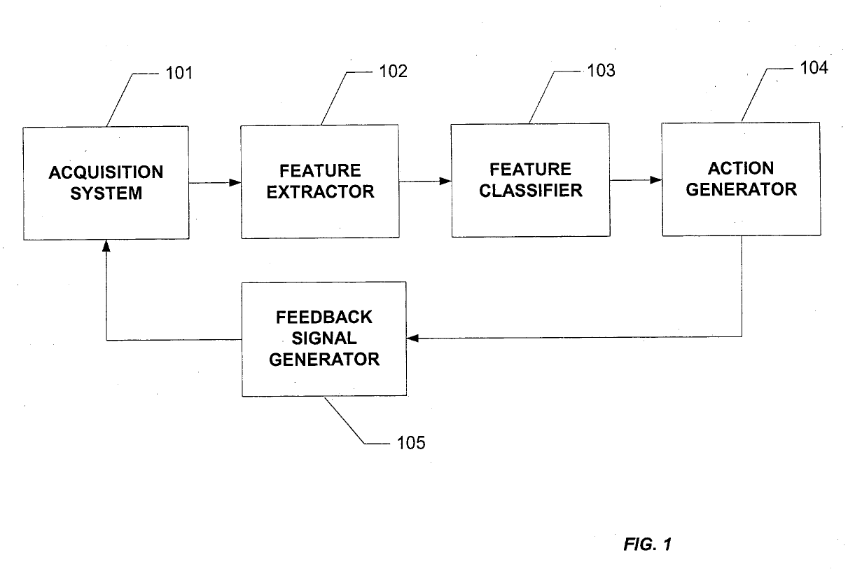

The present invention provides an intelligent supervisory control system, or Neuro-user interface (NUI), and method that utilizes brain signals to control electronic and mechanical resources in an environment, such as in a 3-D PC or console game, a simulation or virtual environment, a cockpit, automobile, home or surgical theatre. The NUI comprises means for acquiring the brain signals of a user or subject, which are converted into a digital stream and mathematically processed to define a representation of the state of the mind or cognitive profile of the user as an output. Incorporating microprocessor-based software and storage facilities, the NUI then dynamically maps the cognitive profile onto multiple functions, which are adaptable for actuating microprocessor commands. In conjunction with other standard input devices such as a mouse, keyboard, or joystick, the intelligent supervisory control of the present invention thus provides the user maximal degrees of freedom for the control of the resources in the environment, or electrical and mechanical devices. With reference to FIGS. 1, 2, 3, 4, 5, 6, 7, and 8, the invented system is now described in detail.

There are seven major components or stages involved in the present invention: 1) recording EEG activity; 2) real-time analog-to-digital conversion; 3) preprocessing and analysis of the data; 4) learning algorithms; 5) pattern recognition; 6) mapping signals to microprocessor commands; and 7) closed-loop feedback.

FIG. 1 is a block diagram representing an intelligent supervisory control system, or neural user interface, of the present invention. An intelligent supervisory control system 100 comprises an acquisition subsystem 101, a feature extractor 102, a feature classifier 103, an action generator 104, and a feedback signal generator. Each the seven stages can be associated with one of the function blocks of FIG. 1, as further described below.

Recording EEG Activity

Acquisition subsystem 101 comprises a sensor as described above. Sensors in the present invention are commonly available, which require a minimum of preparation and electrolytic conducting gel. The placement of these sensors on the scalp of the user or subject is optimized to record the maximal signal with the fewest number of recording sites. Additionally, the system can comprise a high precision, low interference headset, which is easy to put on and operate by non-professionals. This headset utilizes disposable gel-filled inserts as electrodes, with the amplifiers built into the headset. This ensures excellent signal-to-noise and relatively noise- and artifact-free signals. Analog-to digital conversion is performed by dedicated analog-to-digital (A/D) converters that can be built into the headset, and the signal is transmitted, by wire or wireless means, to a remote receiver that is connected to a portable microprocessor. A built-in driver interface permits the data acquisition system to communicate with the remote receiver.

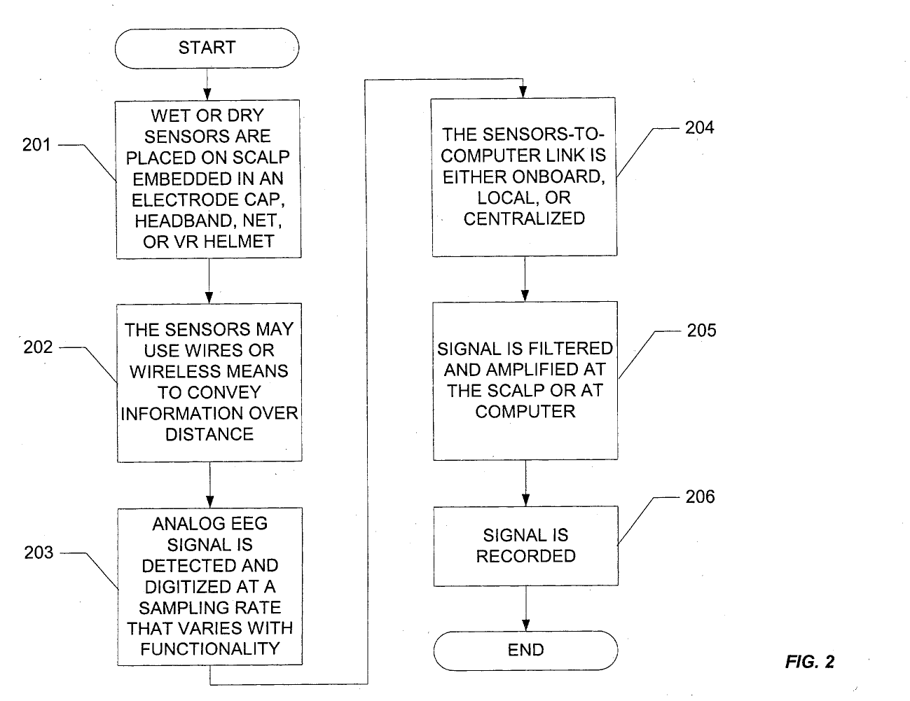

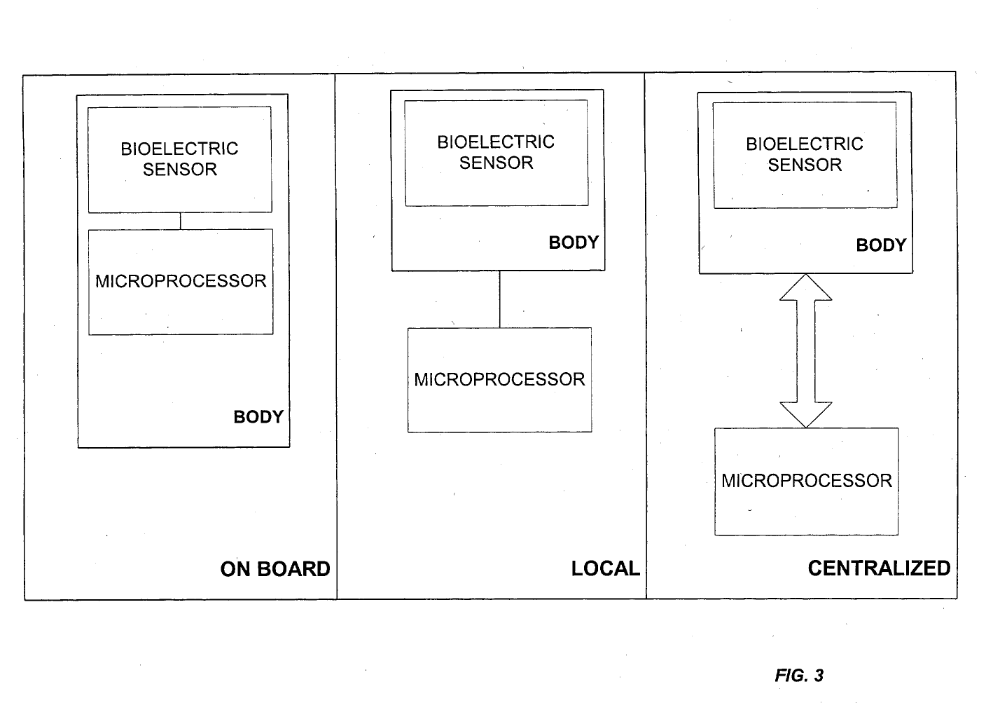

FIG. 2 is a flow diagram of a method for brain signal acquisition in accordance with the methods and systems of the present invention. Wet or dry sensors may be embedded in commercially available conventional electrode caps, headbands, nets, virtual reality (VR) helmets, or other means placed on the head of the user, as shown in step 201 of FIG. 2. In step 202, the sensors use wires and/or wireless means to convey information to the recording microprocessor. The sensors-to-microprocessor link, as shown in FIG. 3, can be onboard (i.e., both sensors and microprocessor are on the body), local (both sensors and microprocessor within a defined distance of each other); or centralized (both sensors and microprocessor at a very large distance from each other).

In step 203, an EEG signal is detected and digitized by an analog to digital board at a sampling rate that varies with functionality. Thus, for example, the use of spontaneous EEG rhythms requires fast sampling rates, while the use of event-related potentials requires slower sampling rates. This is done by means of a recording device such as a microprocessor-based personal computer (step 204). The analog EEG signal is filtered (bandpassed) and amplified (either at the scalp or remotely at the recording microprocessor), and digitized (step 205). In step 206, the signal is recorded.

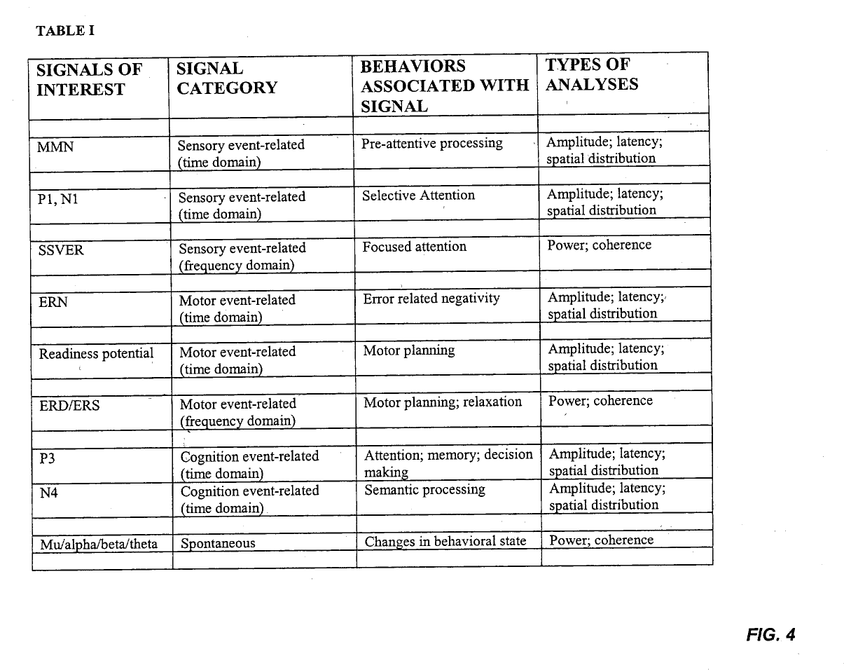

For purposes of illustrating the present invention, recordation and analysis of the spontaneous EEG rhythms and time-locked responses to external events are presented. TABLE I of FIG. 4 lists the variety of brain signals that are captured and used in the present invention to control resources in an user environment. These brain signals are recognizably associated with the dominant behaviors or as identified in TABLE I. In the present invention, these behaviors provide the basis for assessing the cognitive state of the mind or cognitive profile of an individual. Examples of such an user's environment include a 3-D personal computer or console game, a simulation or virtual environment, a cockpit of an airplane, a home, an automobile, and a surgical theatre.

Real-Time Analog-to-Digital Conversion

The present invention provides for the real-time analog-to-digital conversion and analysis of brain signals in stage 2, and incorporates the use of a microprocessor-based scientific software system, which resides in the microprocessor computer for dedicated computerized analysis. The software system includes a library of data analysis routines for processing spontaneous and event-related brain activities, such as digital filtering, signal averaging, real-time power spectrum analysis, and calculation of the ongoing power in different frequency bands. It provides data collection, real time analysis, and delivering of output based on the result of the analysis. The computational output defines the cognitive profile of the user. It is then used to provide feedback information to the subject. In addition, the computational output can also be used to adapt the data analysis/extraction algorithm to best match the incoming data, characterized as adaptive data extraction.

The software system also supports the acquisition of brain signals via analog-to-digital (A/D) converter. Additionally, it provides tools for management of external devices in real-time, with or without simultaneous acquisition of data.

The user's cognitive profile, defined as an output of the present invention, provides two types of outputs for stimulus presentation—digital outputs (for example, turning on/off lights and sound generators, or sending digital information to another microprocessor/program) and analog (graded) outputs via digital-to-analog (D/A) converters. As brain signals are acquired, they are analyzed real time. Based on the result of the analysis, the system triggers a command, such as effecting movement of a cursor.

The system adopts a simple uniform structure for data representation with the same data format for both input and output data, (that is, the raw incoming data and the results of an analysis) to ensure that the output of one computation or process step can be used as an input for another. Thus data already collected and processed can be reused and analyzed in a different way. The system also provides for the export of data in a format that can be used by other microprocessor-based programs to perform independent component analysis or neural net analysis.

Preprocessing and Analysis of the Data (Signal Decomposition)

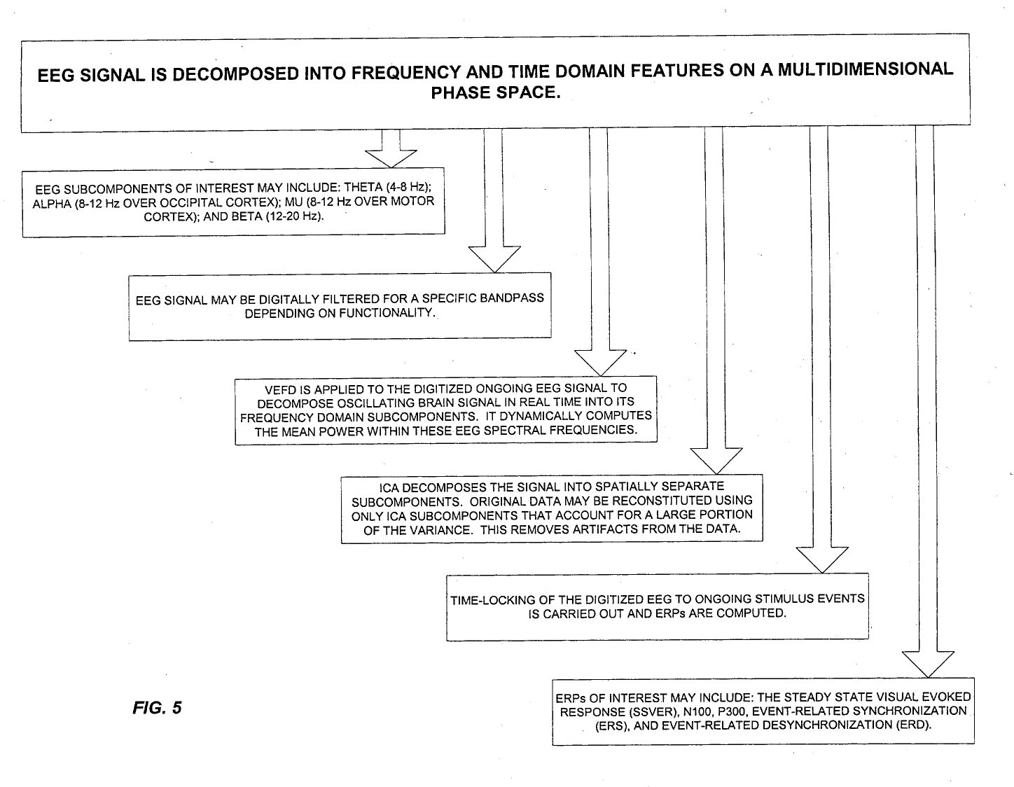

Feature extractor 102 comprises the functionality needed to carry out decomposition of the digitized EEG signal. The digitized EEG signal can be decomposed into frequency and time domain features on a multidimensional phase space in stage 3, as shown diagrammatically in FIG. 5. Frequency and time domain subcomponents are analyzed using a variety of techniques including Variable Epoch Frequency Decomposition (VEFD), Fast Fourier Transform, Event-Related Potentials (ERPs), Independent Component Analysis (ICA), Time-Frequency Expansion, and/or Feature Coherence Analysis. The EEG subcomponents of interest include EEG rhythms, such as mu (7-13 Hz over sensorimotor cortex), theta (4-8 Hz); alpha (8-12 Hz over occipital cortex); and beta (12-20 Hz). They can also include time-locked responses to external events, or event-related potentials, such as Ni, p3, or the steady state visual evoked response (SSVER). The brain signals are digitally filtered for a specific bandpass depending on which of these signals are being used.

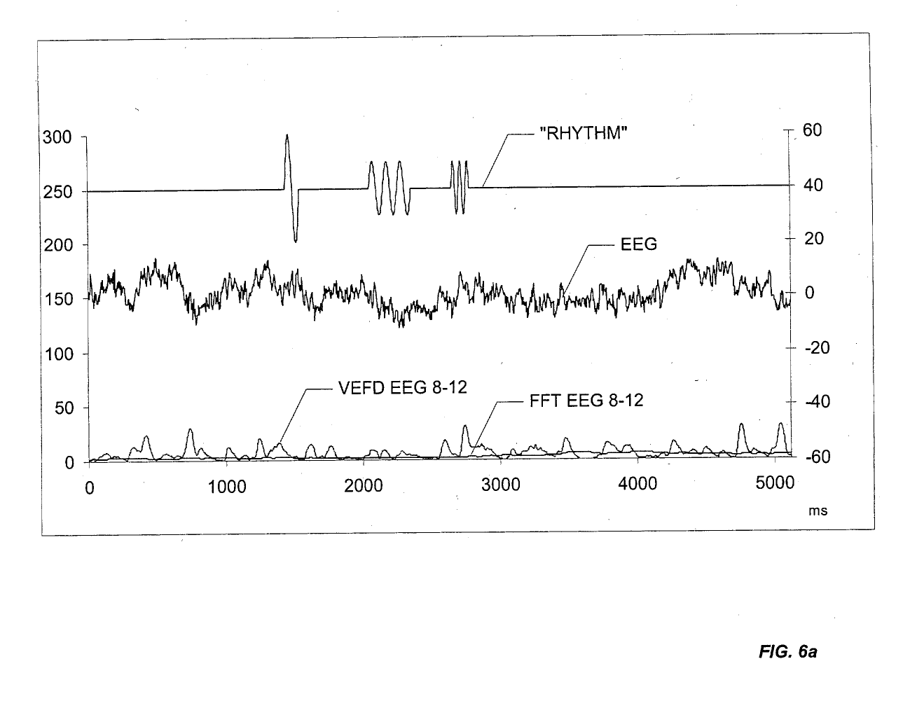

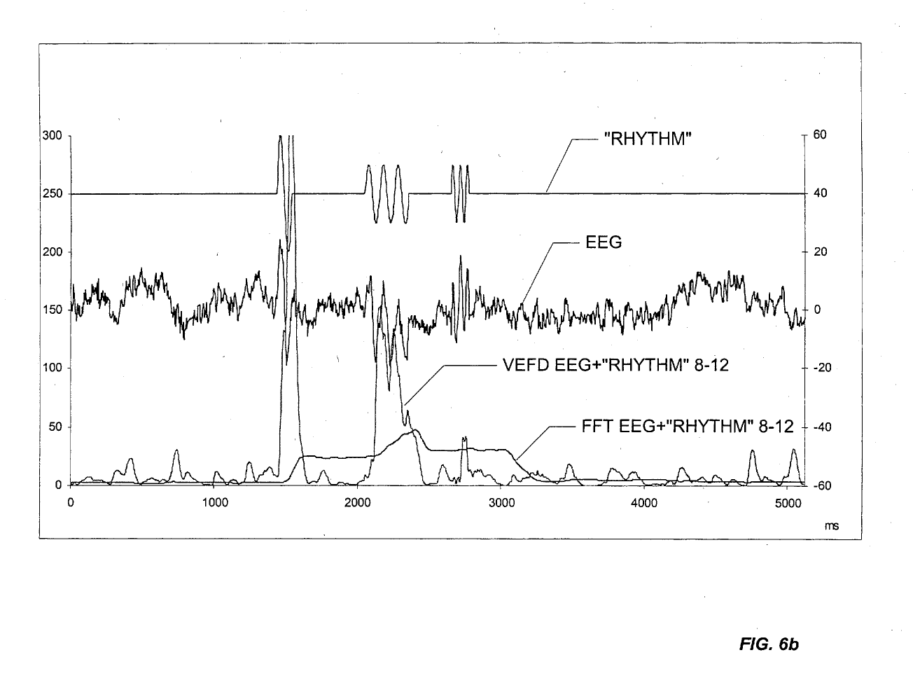

In some applications of the present invention, VEFD, is applied to the digitized ongoing signal to decompose oscillating brain rhythms into their frequency domain subcomponents. For example, in order to determine how tired a user is, the inventive system examines the level of alpha and beta activities in the EEG. In the present invention, the system's ability to detect rhythmic activities is shown in FIG. 6a and FIG. 6b, by using simulated “rhythmic” activity short (1-3 cycles) sine waves of different amplitudes and frequencies, embedded in pre-recorded human EEG, and compared to traditional power spectrum (PS) analysis based on one second epochs of the same signal.

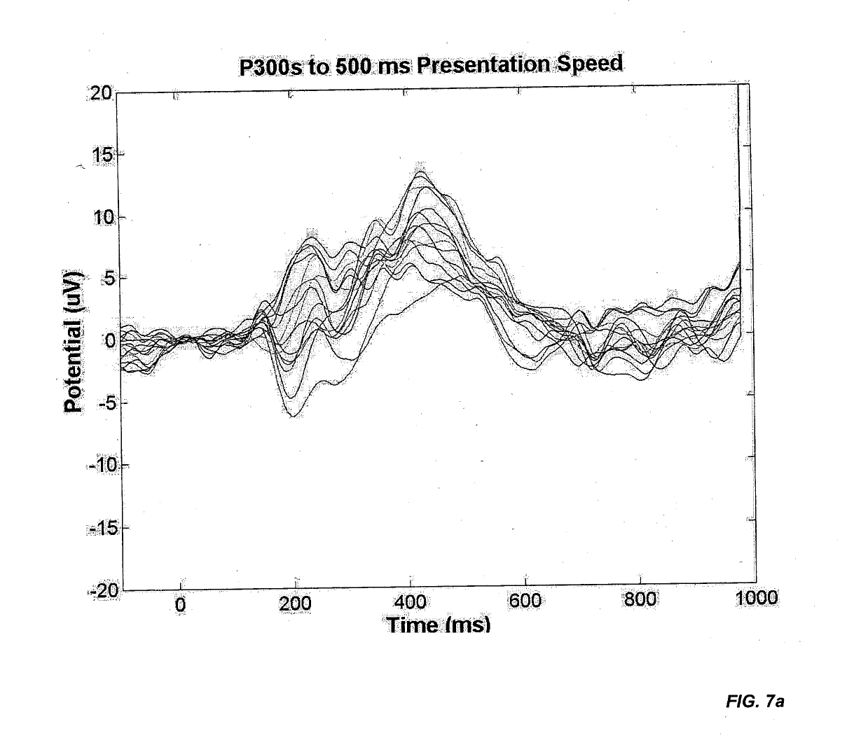

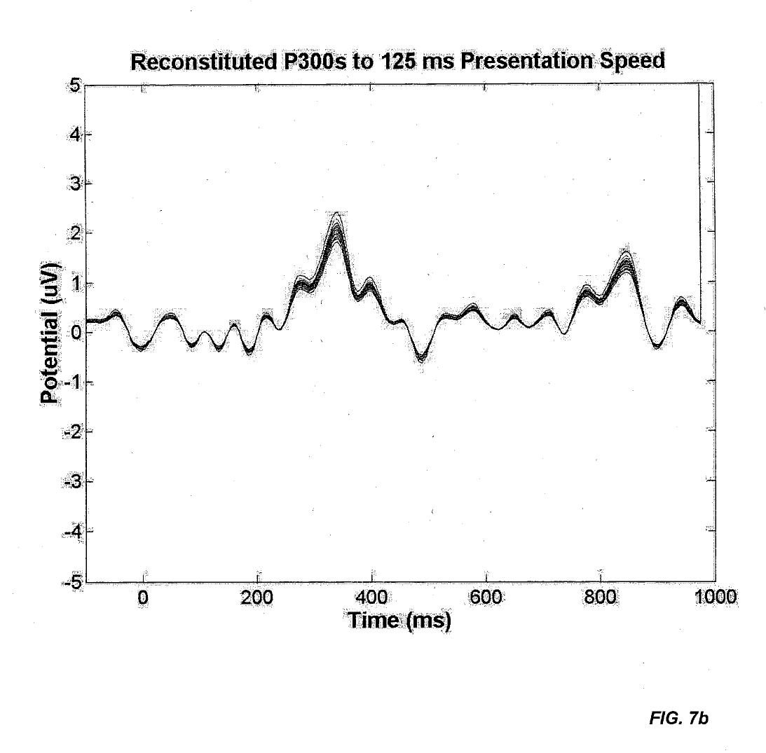

In other applications of the present invention, ICA is applied to decompose the brain signals into spatially separable subcomponents, which maximizes the signal-to-noise response and allows for multiple control signals. Such application enables the reconstitution of the original by using only the ICA subcomponents that account for a large portion of the variance in the signal. Such application removes blinks and eye movement artifacts from the data. Using ICA to “clean” the data in real time increases signal-to-noise, making the relevant signal easier and faster to detect by a pattern recognition system as illustrated in FIG. 7a and FIG. 7b, in which event related potentials are shown before and after application of ICA.

The use of ICA provides solution to the problem of blind source separation, which is analogous to the one posed by recording EEG at multiple sites where the signal at any recording site (be it a satellite, microphone, or electrode) is assumed to consist of a combination of numerous overlapping sources. The locations of these sources are unknown, and the objective is to isolate the contribution of each of these independent sources based on the observed data at each site. Identification of multiple independent control signals in the EEG makes simultaneous control of multiple functions feasible in the present invention. For example, in a game environment it allows an avatar to jump, fire, and signal to others all at the same time.

Learning Algorithms

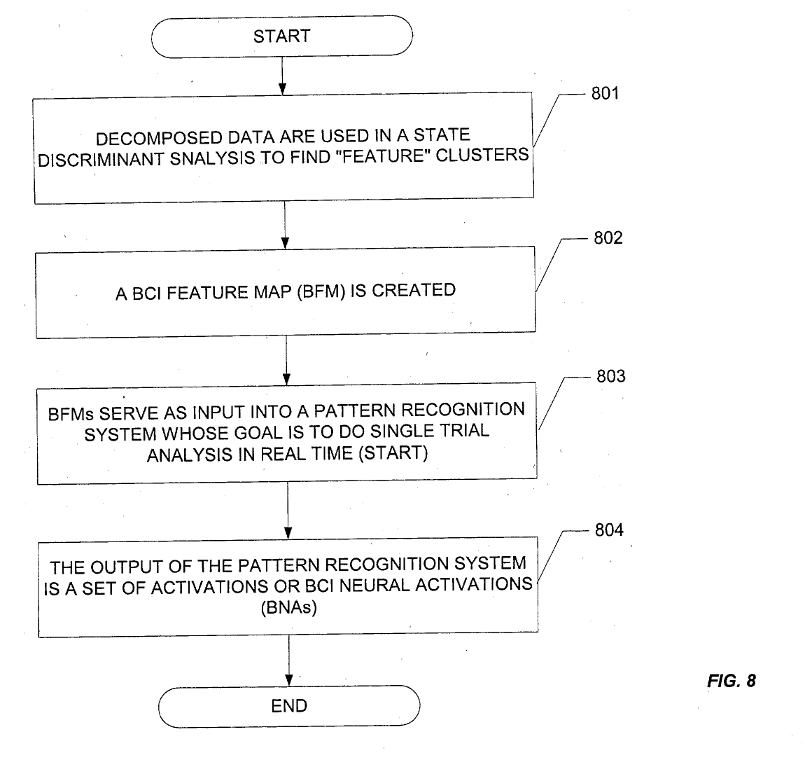

Feature extractor 102 can also be used to identify feature clusters present in the signal. FIG. 8 shows the steps in stage 4 of the present invention whereby decomposed EEG data are resolved by way of a state discriminant analysis to identify “feature” clusters that are most reliably different between different conditions (step 801). Feature clusters represent patterns of electrical activity that occur across the scalp and that are linked to specific motor or non-motor thought patterns. For example, when a user sees a novel image on the screen, a large positive-going voltage can be detected over the middle of the scalp approximately 300 milliseconds after the onset of the novel image. This would be a feature cluster that is identifiable by this type of discriminant analysis. This may be accomplished using: waveform analysis, distribution function analysis, Fuzzy logic, and/or discriminant optimization. In step 802, the outcome of this analysis is the creation of a BCI (Brain Computer Interface) Feature Map (BFM), which is represented as a set of parameters, components, functions, and criteria, which are characteristic of certain aspects of the cognitive state of the mind of the user.

Pattern Recognition

Pattern recognition typically occurs in the feature classifier block 103. As shown in FIG. 8, the present invention incorporates a pattern recognition system as well as the learning algorithms as described above. A plurality of the BFMs are constituted as inputs into a pattern recognition system in stage 5, which may be expressed in the form of a neural network, genetic algorithm, Kohonen network, Fuzzy neural net, or Markov model. The pattern recognition system of in the present invention conducts a single trial analysis in real time (START), as illustrated in step 803. The output of the pattern recognition system is a set of activations or BCI Neural Activations (BNAs), as in step 804. In the present invention, BNAs are derived from adaptive combinations of discriminant brainwave features in space, time, frequency, and phase manipulated to maximize the contrast between the various values of the brainwaves features.

The present invention provides for the classification of patterns of brain activities in real time. Neural networks, or other pattern recognition systems, are used to determine the underlying functional relationship among the various features in the bioelectric signals as they relate to changes in thought patterns. Employing a neural network classifier with modifiable parameters, the present invention identifies the underlying relationships of the different features of the bioelectric signals and the dynamics of the cognitive state of the mind of the users. Also, by supplying the neural network with training sets obtained from recordings on single subjects, the network “learns” the individual patterns of the brain activities and adapts the approach in learning these patterns to correspond to the results obtained by visual inspection of different experts. Such pattern recognition techniques are effective in recognizing complex patterns such as those produced by sensor arrays in actual environmental conditions, as well as from tasks involving categorization of EEG patterns. Thus, the present invention allows for the rapid and reliable recognition and learning of brain activity patterns, which are considered characteristics of the cognitive state of the mind or cognitive profile of the subject, and consistently maps the patterns onto the controls or functions in an environment in a manner that is customized to the subject.

Mapping Signals to Microprocessor Based System Commands and Closed-Loop Feedback

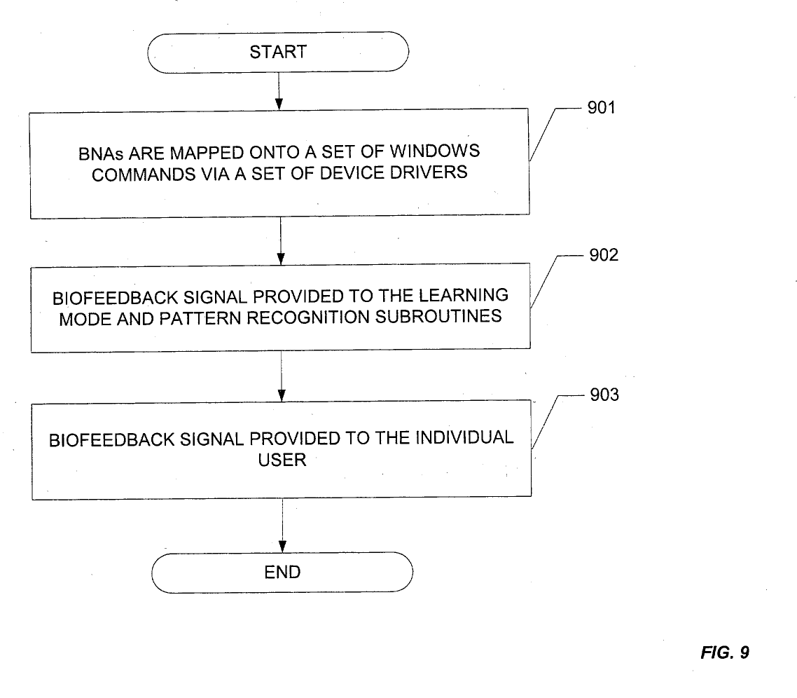

FIG. 9 is a flow diagram of a process for mapping BNAs onto a processor command set. Once a pattern of brain activity is identified, the BNAs are dynamically mapped onto a set of microprocessor-based system commands, as depicted in step 901. By way of examples, the commands may include Windows commands for keyboard command, cursor movement control, file operation, and protocol control. An action generator 104 can be used to implement this type of external control of electromechanical devices.

In step 902, a biofeedback signal is provided to the learning mode and pattern recognition subroutines described previously with respect to FIG. 8. Finally, in step 903, a biofeedback signal is provided to the user. Biofeedback typically occurs within a feedback signal generator 105. The dynamic mapping also enables a user to achieve robust assertion of a desired control in “open-loop” situations where the user does not necessarily need to detect and employ feedback.

In one embodiment of the present invention, the NUI is employed as a controller to control events in an environment, such as a game where movement, weapon switching, weapon firing, and “magical” events can result from controlling the levels of brain activities. In such applications, the NUI is applied in conjunction with game input devices such as keyboards, joysticks, and steering wheels.

The present invention can employ various dynamic mapping techniques, such as Neural Networks (NNs), Markow Models or genetic algorithms in pattern recognition and as robust classifiers. Incorporating such techniques, the present invention has the ability to generalize when making decisions, which involve imprecise input data. Accordingly, the present invention can be applied to control problems where the input variables are measurements used to drive an output actuator, while the network learns the control function.

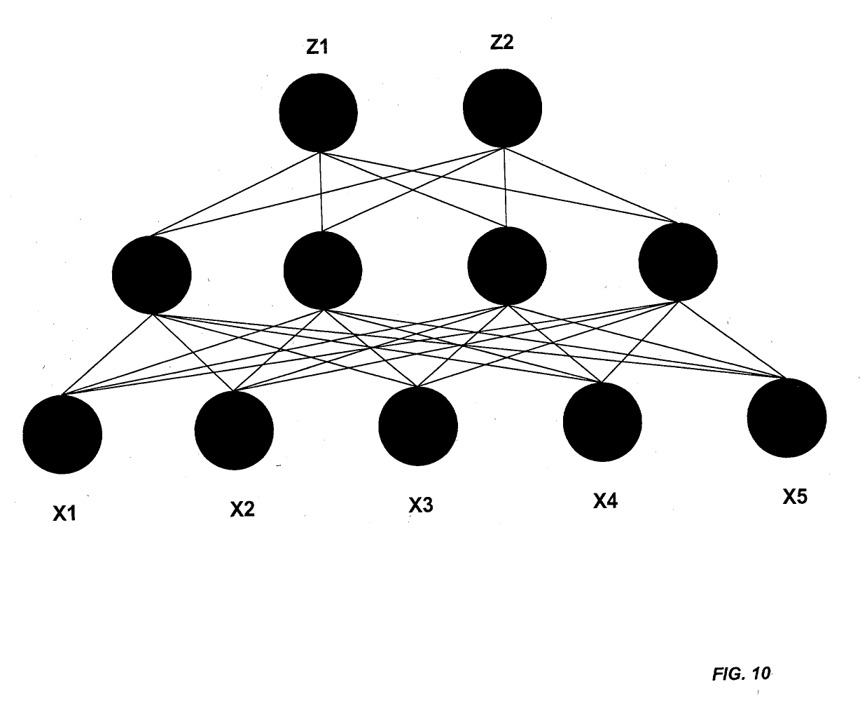

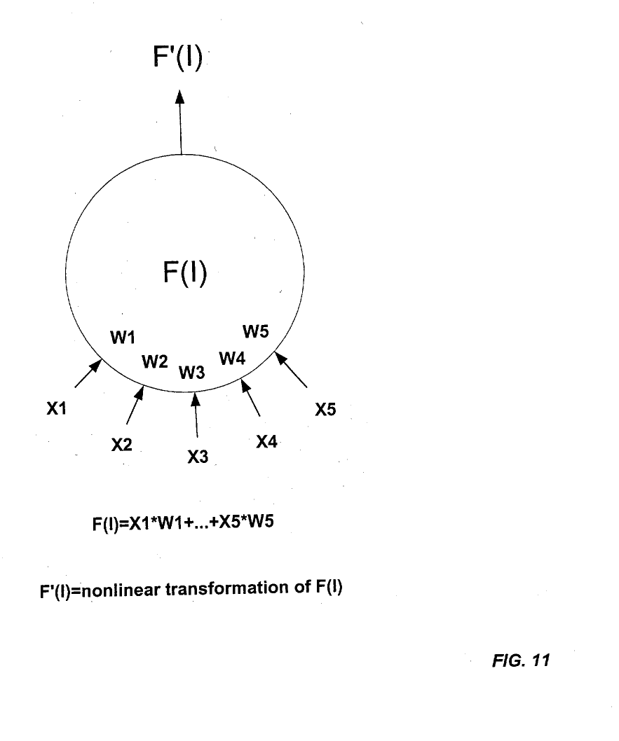

As an example, the structure of a neural network is represented in FIG. 10 where the bottom layer represents the input layer with 5 inputs labeled X1 through X5. The inputs comprise varying levels of three categories of brain signals recorded, which can be extracted from different recording sites. In the middle of the network is the hidden layer, with a variable number of nodes each connected to the inputs. The hidden layer performs much of the work of the network and learns the interdependencies in the model. FIG. 11 illustrates a predefined relationship for the manipulation and translation of the brain signals into the output functions associated with the output layer. By way of an example of a video game, the player may be trying to predict where an enemy soldier may appear (output) based on past appearances, the attentional gaze of the user, and the warning signal from a friendly soldier (input).

The computation associated with the neural network involves a weighted sum: X1 times W1 plus X2 times W2 on through X5 and W5. The weighted sum is performed for each hidden node and each output node and is how interactions are represented in the network. Each summation is then transformed using a nonlinear function before the value is passed on to the next layer.

In operation, the network is repeatedly shown observations from available data related to the problem to be solved, including both inputs (the X1 through X5, as shown in FIG. 10) and the desired outputs (Z1 and Z2). The network attempts to predict the correct output for each set of inputs by gradually reducing the error (back-propagation of error algorithm). There are several different algorithms for accomplishing this (e.g., learning vector quantization and radial basis function, Hopfield, and Kohonen), all of which involve an iterative search for the proper set of weights (the W1-W5) to effect an optimized and accurate prediction of the outputs.

Thus, from the above, it is apparent that the present invention represents a unique, novel, more natural, intuitive, and hands-free means of communicating with and controlling devices or resources in an environment, which takes into account details of non-linear neurodynamics and reflects the user's cognitive state of the mind. It is deployable over diverse areas of human activity, including alternative techniques for system control, increasing the effectiveness of aerospace systems, reducing workload, improving the efficiency within a cockpit, and providing an avenue for hands-free interaction with wearable microprocessors. Furthermore, military, government, and industrial applications include increasing the bandwidth of operator-system interaction, enhancing operator speed and accuracy, confirming that pilots, command officers, air traffic controllers and others in command critical situations have seen important information, improving the operability of military systems, and having the on-demand capability to stealthily communicate with troops in a battlefield. Medical applications include image-guided surgery, passive monitoring of brain disorders, ameliorating attention deficit disorders (ADD) and other attention disorders, as well as providing mobility and communication to the disabled community. Other applications involve monitoring the alertness and cognitive readiness of individuals to ensure they perform their jobs safely and adequately; being able to signal an emergency without the use of voice or hands, and having the ability acquire information and to evaluate the validity, truth or falsity of such information. Consumers can enjoy single- and multi-player games using the NUI, or be involved in advanced biofeedback learning, or in basic computer command and control.

While the above description of the invention is directed to the present embodiments or examples of applications, various modifications and improvements can be made without departing from the spirit and scope of the invention.

What is claimed is:

1. A method for supervisory control over the external environment of a user, comprising:
acquiring a bioelectric signal of a user;
processing the bioelectric signal to define a cognitive profile of the user; and
mapping the cognitive profile onto a set of microprocessor system commands for controlling the external environment of the user.

2. The method of claim 1, wherein the bioelectric signal is an electroencephalogram (EEG) signal.

3. The method of claim 2, further comprising decomposing the EEG signal in to a plurality of signal subcomponents.

4. The method of claim 3, wherein the plurality of signal subcomponents comprises frequency domain subcomponents.

5. The method of claim 4, wherein the frequency domain subcomponents are selected from the group consisting of a mu rhythm, a theta rhythm, an alpha rhythm, and a beta rhythm.

6. The method of claim 3, wherein the plurality of signal subcomponents comprises time domain subcomponents.

7. The method of claim 6, wherein the time domain subcomponents are selected from the group consisting of event-related potentials (ERP) including N1, P3, and steady state visual evoked response (SSVER).

8. The method of claim 3, wherein the processing of the signal further comprises analyzing the signal using one of the group consisting of a variable epoch frequency decomposition (VEFD), a fast Fourier transform (FFT), and independent component analysis (ICA).

9. The method of claim 3, further comprising identifying and classifying feature clusters from the plurality of signal subcomponents.

10. The method of claim 9, further comprising creating a BCI feature map (BFM) from a feature cluster identified through one of the group consisting of a discriminant optimization analysis, a waveform analysis, a distribution function analysis, and fuzzy logic.

11. The method of claim 10, further comprising performing real-time pattern recognition on the BFM to produce a set of BCI neural activations (BNAs).

12. The method of claim 1, further comprising sending a feedback signal to the user.

13. A supervisory system for controlling the external environment of an user, comprising:
signal acquisition means for acquiring a bioelectric signal of a user, the bioelectric signal comprising an electroencephalogram (EEG) rhythm; and
a controller in communication with the signal acquisition means for controlling the external environment of a user, wherein the controller comprises:
a processor for processing the bioelectric signal to define a cognitive profile; and
a mapping algorithm for mapping the cognitive profile onto a set of microprocessor system commands for controlling the external environment of the user.

14. The system of claim 13, which further comprises a feature extractor for decomposing the digitized bioelectric signal in to a plurality of signal subcomponents.

15. The system of claim 14, wherein the plurality of signal subcomponents comprises frequency domain subcomponents.

16. The system of claim 15, wherein the frequency domain subcomponents are selected from the group consisting of a mu rhythm, a theta rhythm, an alpha rhythm, and a beta rhythm.

17. The system of claim 14, wherein the plurality of signal subcomponents comprises time domain subcomponents.

18. The system of claim 17, wherein the time domain subcomponents are selected from the group consisting of event-related potentials (ERP) including N1, P3, and steady state visual evoked response (SSVER).

19. The system of claim 13, wherein the processor analyzes the bioelectric signal using one of the group consisting of a variable epoch frequency decomposition (VEFD), a fast Fourier transform (FFT), and independent component analysis (ICA).

20. The system of claim 14, wherein the controller further comprises a feature classifier for identifying feature clusters from the signal subcomponents.

21. The system of claim 20, wherein the feature classifier creates a BCI feature map (BFM) from a feature cluster identified through one of the group consisting of a discriminant optimization analysis, a waveform analysis, a distribution function analysis, and fuzzy logic.

22. The system of claim 20, wherein the feature classifier performs real-time pattern recognition on the BFM to produce a set of BCI neural activations (BNAs).

23. The system of claim 13, further comprising a feedback signal generator for sending a feedback signal to the user.

24. The system of claim 13, further comprising an action generator coupled to the controller for controlling the movement of an electromechanical device in the external environment.

25. The system of claim 13, wherein the signal acquisition means is a sensor.

26. The system of claim 13, wherein the processor comprises a central processing unit (CPU).

27. The system of claim 13, wherein the processor comprises a software control program.
